(19) United States
(12) Patent Application Publication
(10) Pub. No.: US 2026/0020806 A1
(43) Pub. Date: Jan. 22, 2026

(54) IMPLANTABLE EMBEDDED SYSTEMS AND RELATED MANUFACTURING METHODS

(71) Applicant: Blackrock Microsystems, Inc., Salt Lake City, UT (US)

(72) Inventors: Florian SOLZBACHER, Salt Lake City, UT (US); Moritz Michael LEBER, Salt Lake City, UT (US); Daniel Richard POWELL, Salt Lake City, UT (US); Robert Kyle FRANKLIN, IV, Centerville, UT (US); Sandeep NEGI, Salt Lake City, UT (US); Sherman Perry WIEBE, Brussels (BE)

(21) Appl. No.: 19/272,695

(22) Filed: Jul. 17, 2025

Related U.S. Application Data
(60) Provisional application No. 63/673,018, filed on Jul. 18, 2024.

(51) Int. Cl. A61B 5/293; A61B 5/268; H01L 21/306; H01L 21/3065; H01L 21/311; H01L 21/683; H01L 21/768

(52) U.S. Cl. CPC A61B 5/293; A61B 5/268; H01L 21/30625; H01L 21/3065; H01L 21/31116; H01L 21/6835; H01L 21/76885; A61B 2562/046; A61B 2562/125

(57) ABSTRACT

Embodiments described herein include a method for manufacturing a neural implant including patterning a circuit-bearing substrate to produce a first intermediate structure having a plurality of electrode contacts disposed on a first side thereof. The method can include applying an encapsulation layer to the first side of the first intermediate structure to produce a second intermediate structure and coupling a first surface of the second intermediate structure to a carrier, the first surface of the second intermediate structure including the encapsulating layer. The method can include thinning the second intermediate structure to produce a third intermediate structure and forming a plurality of recesses in a portion of the third intermediate structure to produce a fourth intermediate structure. The method can include releasing the carrier from the fourth intermediate structure to produce the neural implant.

### CROSS-REFERENCE TO RELATED APPLICATIONS

This application claims priority to and the benefit of U.S. Provisional Application No. 63/673,018, filed Jul. 18, 2024, and titled, “Implantable Embedded Systems and Related Manufacturing Methods,” the disclosure of which is incorporated by reference herein in its entirety.

### TECHNICAL FIELD

Embodiments herein relate to methods for manufacturing implantable neural devices embedded in/on a thinned circuit-bearing substrate.

### BACKGROUND

Common implantable neural devices include electrode contacts configured to be disposed on or in a patient's brain, along with support electronics (e.g., a power supply, communication interface(s), etc.) that are typically disposed in a hermetically sealed metal that can be implanted separately from the electrode contacts (e.g., in a chest cavity of the patient). The electrode contacts may be electrically connected to the support electronics using wires or leads. The use of wired connections through the body and the use of welding to hermetically seal the metal can result in or increase the likelihood of failure(s) in the associated neural device system, and can increase manufacturing complexity. Additionally, implantable neural devices manufactured in this manner may lack longevity, require invasive implantation procedures, rely on more materials, and/or incur higher costs to manufacture.

### SUMMARY

One or more embodiments described herein relate to a method for manufacturing a neural implant. The method includes patterning a circuit-bearing substrate to produce a first intermediate structure having a plurality of electrode contacts disposed on a first side thereof. The method also includes applying an encapsulation layer to the first side of the first intermediate structure to produce a second intermediate structure, and coupling a first surface of the second intermediate structure to a carrier, the first surface of the second intermediate structure including the encapsulating layer. The method also includes thinning the second intermediate structure along a direction that extends from a second surface of the second intermediate structure toward the first surface of the second intermediate structure, the second surface opposite the first surface, to produce a third intermediate structure. The method also includes forming a plurality of recesses in a portion of the third intermediate structure, the plurality of recesses having a predefined pattern, to produce a fourth intermediate structure, and releasing the carrier from the fourth intermediate structure to produce the neural implant. In some such implementations, the neural implant does not include a hermetic seal.

In some aspects, applying the encapsulation layer includes depositing an encapsulant followed by patterning the deposited encapsulant. In some aspects, patterning the deposited encapsulant includes plasma etching. In some aspects, the plurality of electrode contacts includes at least one of tantalum, tantalum oxide (Ta₂O₅), titanium, titanium nitride, PEDOT, carbon nanotubes, gold, platinum, iridium, iridium oxide, ruthenium oxide, or an alloy(s) (e.g., an alloy that includes any of the foregoing materials). In some aspects, the encapsulation layer includes at least one of silicon carbide (SiC), doped SiC, polyimide, alumina (Al₂O₃), liquid crystal polymer (LCP), or parylene-C. In some aspects, the SiC includes at least one of amorphous SiC or polycrystalline SiC. In some aspects, the encapsulation layer is a first encapsulation layer including a first material, the method further including applying a second encapsulation layer to the first encapsulation layer, the second encapsulation layer including a second material different from the first material. In some aspects the second material includes an oxide. In some aspects, the second material includes silicon dioxide (SiO2). In some aspects, the carrier comprises at least one of quartz, glass, metal, silicon, or ceramic.

In some aspects, thinning the second intermediate structure includes at least one of mechanically or chemically thinning the second intermediate structure. In some aspects, thinning the second intermediate structure includes performing at least one of chemical mechanical planarization (CMP) or reactive-ion etching (RIE) to remove silicon from the second surface of the second intermediate structure. In some aspects, thinning the second intermediate structure is performed until a predefined thickness is reached, by one of (1) performing at least one of mechanically or chemically thinning for a predetermined amount of time, or (2) using an etch stop layer. In some aspects, the predefined thickness is achieved by at least one of: (1) performing the at least one of mechanically or chemically thinning the second intermediate structure for a predetermined amount of time, or (2) using an etch stop layer. In some aspects, patterning the circuit-bearing structure includes performing metallization of the circuit-bearing substrate. In some aspects, the metallization includes sputtering, and the patterning includes at least one of lithography or a lift-off process. In some aspects, the neural implant is configured to wirelessly receive at least one of power or data. In some aspects, the encapsulation layer is a first encapsulation layer, the method further comprising applying a second encapsulation layer to the fourth intermediate structure prior to releasing the carrier from the fourth intermediate structure. In some aspects, the second encapsulation layer coats the fourth intermediate structure such that at least a portion of the fourth intermediate structure is not exposed to an outside environment. In some aspects, the second encapsulation layer includes at least one of silicon carbide (SIC), polyimide, alumina, or parylene-C. In some aspects, the encapsulation layer is a first encapsulation layer, the method further comprising, prior to releasing the carrier from the fourth intermediate structure, applying a second encapsulation layer to the fourth intermediate structure and patterning the second encapsulation layer. In some aspects, the carrier includes a silicon carbide coating, and the coupling of the first surface of the second intermediate structure to the carrier further includes at least one of bonding the first surface of the second intermediate structure to the silicon carbide coating using anodic bonding or depositing the silicon carbide on the first surface of the second intermediate structure using chemical vapor deposition (CVD). In some aspects, the forming the plurality of recesses in the portion of the third intermediate structure includes etching each recess from the plurality of recesses from the second surface of the third intermediate structure toward the first surface. In some aspects, each of the plurality of recesses extend through at least a portion of the circuit-bearing substrate. In some aspects, the plurality of recesses are arranged in a space between adjacent or neighboring electrode contacts from the plurality of electrode contacts.

### BRIEF DESCRIPTION OF THE DRAWINGS

FIG. 1 includes a schematic block diagram of an implantable neural device, according to embodiments.

FIG. 2A is a flow chart diagram of a first example method for manufacturing an implantable neural device, according to embodiments.

FIG. 2B is a flow chart diagram of a second example method for manufacturing an implantable neural device, according to embodiments.

FIGS. 3A-3G illustrate a third example method for manufacturing an implantable neural device, according to embodiments.

FIGS. 4A-4H illustrate a fourth example method for manufacturing an implantable neural device, according to embodiments.

FIG. 5 shows an example implantable neural device manufactured using the method of FIGS. 3A-3G.

### DETAILED DESCRIPTION

Implantable neural devices typically include one or more electrode contacts electrically connected via one or more leads to support electronics such as, for example, an implantable pulse generator (IPG), a communication interface(s), and/or a power supply. The one or more electrode contacts may be disposed on a surface of a cortex of a patient and/or implanted in the brain of the patient. The one or more electrode contacts may be configured to record brain activity and/or provide stimulation to a targeted brain region of the patient. The IPG, communication interface(s), and power supply are typically disposed in a housing (e.g., a titanium or ceramic can) that is hermetically sealed and disposed in a portion of the body, such as a chest cavity of the patient. The manufacturing process for such implantable neural devices can be complex and expensive, as there are many potential points of failure in the device due to use of wired leads and welding, for example. Furthermore, current neural device systems are bulky and require invasive surgeries for implantation of the electronics housing. Disadvantages of current systems include, for example, (1) connection issues between the electrodes and the electronics housing (e.g., broken leads); (2) degradation of welds hermetically sealing the electronics housing over time; (3) separate implantation locations of electrodes and accompanying electronics; (4) difficult explantation; (5) a limited number of electrical leads and feedthroughs from an electrode(s) to an interior of the associated electronics enclosure; and (6) high cost and complexity of manufacturing.

Some embodiments described herein relate to an implantable neural device that is at least in part monolithically formed using a thinned substrate (e.g., a silicon-based wafer). As used herein, “monolithically formed” refers to the formation of an integrated circuit using a single semiconductor substrate or base material, and then selectively processing it with subtractive and/or additive manufacturing steps to create various active or inactive devices or features on it. In a monolithic implementation, all functional components of the device—such as the electrode array, ASIC, and wireless communication IC—are integrated onto a single substrate through a unified fabrication process, eliminating the need for post-fabrication assembly. By contrast, a non-monolithic (or heterogeneous) approach involves fabricating each component on separate substrates, which are subsequently assembled to form the complete device. Depending on the specific embodiment or implementation, implantable neural devices of the present disclosure may be fully monolithically formed, partially monolithically formed, or not monolithically formed.

In some embodiments, the implantable neural device may include at least a portion of electronics embedded in and/or on the substrate. In some embodiments, the implantable neural device may include all electronics embedded in and/or on the substrate. For example, the implantable neural device may include at least one electrode contact, signal processing circuitry, a memory, a processor, a power source, and/or a communication interface (e.g., a wireless communication interface(s)) integrated into the substrate. In some embodiments, the implantable neural device may be configured to be disposed on a surface of a brain of a patient. The implantable neural device may be configured to record brain activity from a portion of the brain and process and/or analyze the recorded brain activity using circuitry integrated into the substrate. In some embodiments, the substrate may be thinned to increase flexibility of the neural device such that the neural device can fit, track, mimic, be substantially conformal to, and/or generally be shaped to a curvature of the brain. Alternatively, the substrate may be dimensioned (e.g., may have a predefined geometry) or otherwise be configured such that it is not flexible, but can still fit, track, mimic, be substantially conformal to, and/or generally be shaped to be stably positioned on a surface (e.g., a curvature) of the brain. In one or more embodiments, the neural device can be designed, engineered, dimensioned and/or otherwise configured such that a pressure on, deformation of, or irritation of the tissue in contact with the device (e.g., brain tissue) is minimized. Alternatively, an implantable neural device may include one or more areas/regions thereof that are flexible, such that they are substantially conformal to brain tissue and/or reduce tissue response, while also including one or more areas/regions thereof that are not flexible (e.g., thinned but not sufficiently thinned to cause or increase flexibility), so as to avoid negatively impacting electronic properties associated with electronics positioned thereon and/or embedded therein. In some implementations, thin film encapsulation based processes of the present disclosure facilitate the accommodation/inclusion of a greater number of electrical connections than would otherwise be achievable using known methods. Alternatively or in addition, in some implementations, thin film encapsulation based processes of the present disclosure result in fully embedded systems that do not use complex interconnects such as feedthrough pins or ball seal interconnectors, and whose power and data transfer can be managed wirelessly.

In some embodiments, the substrate may be encapsulated by one or more encapsulation materials (also referred to as “encapsulants”) to protect the electronics from degradation due to contact with biological material. Exposure to biological materials can, for example, etch encapsulation materials over time. The system, devices, and methods described herein provide for the application of encapsulants that may be configured to withstand such etching over a long period of time with target lifetimes potentially exceeding 10 years and beyond. In some embodiments, the encapsulation material may include silicon dioxide, silicon carbide, polyimide, and/or parylene-C to protect the electronics from degradation due to biological materials. One or more methods described herein relate to the manufacture of an implantable neural device by depositing pinhole-free (or substantially pinhole-free) encapsulation layers onto a substrate. In other words, the method may include depositing encapsulation layers substantially free of openings or pores (e.g., between the external environment and the substrate or electronics) that can cause or constitute defects in the encapsulation layer(s). Because the electronics of the neural device are not disposed in a housing (e.g., the metal can), the neural device is not hermitically sealed, rather the encapsulation layers may prevent or reduce degradation of the electronics from exposure to biological materials.

In some embodiments, a process for manufacturing the implantable neural device may include forming a plurality of electrode contacts on a first side of a circuit-bearing substrate. For example, a silicon-based wafer may include existing integrated microelectronics, and patterning may be performed on the silicon-based wafer to form electrode contacts on the silicon-based wafer. In some embodiments, the process can include encapsulating at least a portion of the circuit-bearing structure including the plurality of electrode contacts. The process can include thinning the silicon (e.g., a back side thereof) by a predefined amount to increase a flexibility of the neural device. In some embodiments, the manufacturing process may include forming a plurality of recesses in the circuit-bearing structure and/or silicon, the plurality of recesses having a predefined pattern. The plurality of recesses may further increase a flexibility of the neural device and/or may improve a biocompatibility of the neural device. In some embodiments, the neural implant may be coated (e.g., dip coated) with a biocompatible material that reduces the inflammatory response and promotes healing after implantation.

One or more embodiments described herein provide advantages over known neural device systems, e.g., including reduced complexity of manufacturing of implantable neural devices, reduced likelihood of failure(s) due to connection points in the neural device, integrating all electronics into a single, compact device, and increasing case of implantation and explantation of the neural device.

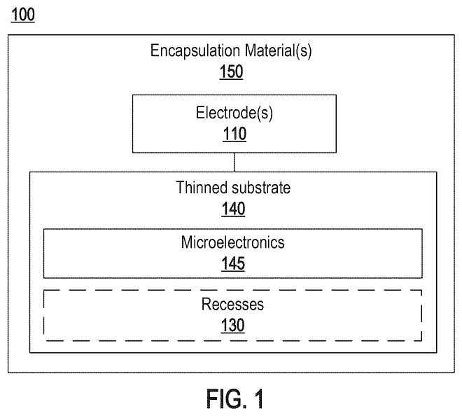

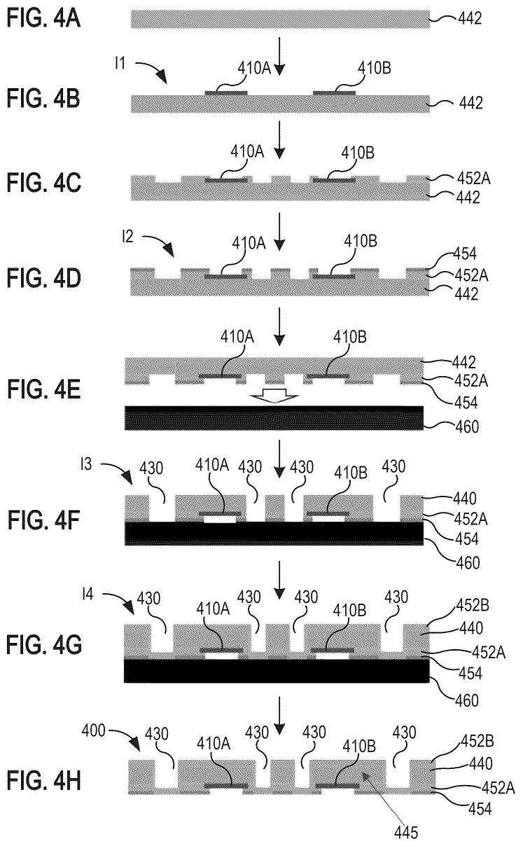

FIG. 1 includes a schematic block diagram of a neural device 100, according to embodiments. As shown, the neural device 100 includes a thinned substrate 140 including microelectronics 145 integrated therein. One or more electrodes 110 may be disposed on the thinned substrate 140. In some embodiments, the one or more electrodes 110 may be disposed on a first side of the thinned substrate 140. In some embodiments, the thinned substrate 140 may optionally define, or include defined therein, one or more recesses 130. The one or more recesses 130 may be defined on the first side of the thinned substrate 140 and/or on a second side of the thinned substrate 140 opposite the first side of the thinned substrate 140. For example, in some embodiments, the one or more recesses 130 may be formed by etching the second side of the thinned substrate 140 (e.g., the side opposite the electrode contacts). In some embodiments, the one or more recesses 130 may not extend entirely from the first side to the second side. In some embodiments, the one or more recesses 130 may extend through an entirety of the thinned substrate 140. One or more encapsulation material(s) 150 may be disposed on at least a portion of the thinned substrate 140 (e.g., including a surface(s) of the recesses 130) and the electrode(s) 110. In some embodiments, the encapsulation material(s) 150 may form a layer that encloses/covers/encapsulates at least a portion of the sidewall and/or bottom (if applicable) of each recess 130 (e.g., as seen in FIG. 4G-4H).

In some embodiments, the thinned substrate 140 may be a thinned silicon-based wafer. The thinned silicon-based wafer may be received from a foundry and may include existing microelectronics 145 disposed therein. The microelectronics 145 may include analog and/or digital signal processing circuitry (e.g., amplifier(s), filter(s), spike detector(s), spike classifier(s), actuator(s), etc.) for collecting and/or analyzing recorded brain signals and/or transmitting the recorded brain signals to an external device. In some embodiments, the microelectronics 145 may include analog and/or digital electronics for applying electrical pulses to stimulate neurons. For example, the microelectronics 145 may include a pulse generator for activating the electrodes to stimulate neurons. In some embodiments, the microelectronics 145 may include a communication interface (e.g., an antenna, optionally with electronics suitable to drive/use the antenna) for wireless communication of signals from the neural implant 100 to an external device. In some embodiments, the microelectronics 145 may include a power source (e.g., a wireless power source such as an inductive charger, resonant inductive charger, or radiofrequency charger) for powering the neural device 100. In some embodiments, the neural implant 100 may be configured to wirelessly receive or transmit at least one of power or data.

In some embodiments, the electrode(s) 110 may include a conductive material that is biocompatible. In some embodiments, the electrode(s) 110 may include a conductive material, such as, for example, tantalum/Ta₂O₅, titanium, PEDOT, platinum, iridium, iridium oxide, gold, tin oxide, carbon, carbon nanotubes, graphene, silver, silver chloride, stainless steel, tungsten, conductive polymers, alloys, or any suitable combination thereof. In some embodiments, the electrode(s) 110 may include platinum, iridium, and/or iridium oxide. In some embodiments, the neural device 100 may include an electrode density in a range of about 1 electrode per 35 square micrometers (μm²) or about 1,000 electrodes per square millimeter (mm²). In some embodiments, the neural device 100 may include an electrode density in a range of about 1 electrode per mm² to about 1,500 electrodes per mm², or about 500 electrodes per mm² to about 1,500 electrodes per mm², inclusive of all ranges and subranges therebetween. In some embodiments, a plurality of electrodes may be connected or combined into a single (one) electrode contact or multiple electrode contacts (e.g., a quantity of which may be less than a number of electrodes in the plurality of electrodes).

In some embodiments, the encapsulation material(s) 150 may include a biocompatible polymer or glass. In some embodiments, the encapsulation material(s) 150 may include one or more insulative materials. In some embodiments, the encapsulation material(s) 150 may include a silicon-based polymer and/or an oxide. In some embodiments, the encapsulation material(s) may include silicon carbide, polyimide (PI), parylene-C, silicon dioxide, liquid-crystal polymer (LCP), silicone, photoresist (e.g., SU-8), alumina, ceramic-polymer composites, compounds such as polymers with silicon carbide (SiC) partially diffused therein, doped SiC, or any other suitable biocompatible material. In some embodiments, the encapsulation material(s) 150 may have a thickness in a range of 10 nm to 50 μm, inclusive of all ranges and subranges therebetween. In some embodiments, the encapsulation material(s) 150 may be disposed on the thinned substrate 140 such that the electrode(s) 110 are at least partially exposed. In some embodiments, the encapsulation material(s) 150 may be disposed on the first side of the thinned substrate 140 including the electrode(s) 110. In some embodiments, the encapsulation material(s) 150 may be disposed on the first side and a second side opposite the first side of the thinned substrate 140. In some embodiments, the encapsulation material(s) 150 disposed on the thinned substrate 140 in one or more layers. In some embodiments, the encapsulation material(s) 150 may include a first layer including a first material. In some embodiments, the encapsulation material(s) 150 may include more than one material. In some embodiments, the encapsulation material(s) 150 may include the first layer including the first material and a second layer disposed on the first layer and including a second material. In some embodiments, the first material and the second material may be different materials. In some embodiments, the thinned substrate 140 may include the first layer including the first material on the first side and a second layer disposed on the second side opposite the first side including the first material. In some embodiments, the first side of the thinned substrate 140 may include the first layer of the first material and a second layer of the second material, and the second side of the thinned substrate 140 may include a third layer of the first material. In some embodiments, the encapsulation material(s) 150 (e.g., the first material and/or the second material) may include any suitable material such as, for example, nitrides, oxides, polymers, glass. In some embodiments, the first material may include parylene-C, polyimide, and/or silicon carbide. In some embodiments, the second layer may include silicon dioxide. In some embodiments, the encapsulation material(s) 150 may be applied to all surfaces of the thinned substrate 140 such that no portion of the thinned substrate 140 is exposed to an outside environment (e.g., a biological environment inside a patient). In some embodiments, the encapsulation material(s) 150 may be applied to one or more surfaces of the thinned substrate 140 such that at least a portion of the thinned substrate 140 is not exposed to an outside environment (e.g., a biological environment inside a patient). In some embodiments, an entire outer surface of the thinned substrate 140 may be covered by a first layer of the first material, and a portion of the first layer may be covered by a second layer of a second material (e.g., different than the first material). In some embodiments, portions of the first layer or the second layer may be removed to expose the one or more electrode contacts. In some embodiments, the encapsulation material(s) 150 may include a plurality of layers of the same material.

In some embodiments, the recesses 130 may be defined in the thinned substrate 140 in a predefined pattern such that the neural device 100 includes a suitable flexibility and can conform to an irregular three-dimensional surface (e.g., gyri and/or sulci of a portion of a brain of a patient). Non-limiting examples of recessed structures (and/or of the manufacture thereof) compatible with embodiments of the present disclosure can be found in U.S. Patent Application Publication No. 2023/0064374, published Mar. 2, 2023 and titled “Apparatus and Methods to Provide a Scalable and Flexible High Channel Density Neural Interface,” the entirety of which is incorporated by reference herein.

The predefined pattern can be any suitable pattern including that of a mesh or a lace geometry. In some instances, a predefined pattern of recesses 130 can include repeating recesses or recess units of one or more shapes forming a lattice structure generating a mesh or lace geometry. In some embodiments, the pattern can include recesses 130 having repeating units of one or more shapes. In some embodiments, the pattern of recesses 130 can include repeating recess units of any suitable shape (e.g., square, rectangle, circle, oval, polygon and/or the like). In some embodiments, the pattern of recesses 130 can include a tessellated pattern of recesses 130 (also referred to as recess units) covering a surface area of a substrate (e.g., a substrate formed by a planar portion of the neural device 100. In some embodiments, the neural device 100 can include recesses 130 according to a single predefined pattern. The pattern may be selected based on a desired pattern of stimulating and/or recording neural activity in a tissue upon implantation. In some instances, the pattern may be selected based on a physiology of a target tissue (e.g., a structure of the tissue, a foreign body response expected upon implantation, a degree of access to the tissue that is desired, a target implementation of the neural apparatus (e.g., a treatment plan, a diagnostic plan, and/or the like), etc.) In some embodiments, the set of recesses 130 can form a lattice structure that includes nodes. In some embodiments, the neural device 100 can include recesses 130 according to multiple predefined patterns.

In some embodiments, the recesses 130 (e.g., cavities, openings, perforations, holes, through-holes, etc.) may be configured to improve biocompatibility of the neural device 100 by reducing a surface area of the neural device 100 and/or reduce an area of contact between the neural device 100 and biological tissue. In some embodiments, the recesses 130 may enable cross flow of biological fluids across the neural device 100. In this way, the recesses 130 may improve biocompatibility by mediating improved clearance of potentially cytotoxic factors released from cells (e.g., immune cells, and/or injured cells from iatrogenic impact of implantation, etc.). In some embodiments, the neural device 100 may include a number of recesses 130 in a range of about 1 recess per mm² to about 300 recesses per mm², inclusive of all ranges and subranges therebetween. In some embodiments, the neural device 100 may include a number of recesses 130 in a range of about 4 recesses per mm². In other embodiments, the neural device 100 may include a number of recesses 130 in a range of about 8 recesses per mm². In some embodiments, the recesses may have a pitch of about 400 μm, center-to-center, between recesses.

In some embodiments, the neural device 100 may be coated with a coating that reduces the inflammatory response and promotes healing after implantation. In some embodiments, the coating may include a biocompatible material such as an amniotic membrane, a coating that uses or includes an amniotic membrane ingredient, an anti-inflammatory material, or a gel material. In some embodiments, the coating may be applied to the neural device 100 in a bath and/or by dip-coating. In some embodiments, the neural device 100 may be moved through (e.g., punched through, pushed through, etc.) a dry sheet of the biocompatible material, and the dry sheet may then be hydrated (e.g., with water) such that the coating conforms to the neural device 100. In still other embodiments, the coating may be applied using one or more of: molding, spray coating, 3D printing, holographic lithography for gel forming, or lamination. In some embodiments, the neural device 100 optionally includes one or more protrusions (e.g., penetrating beam structures), with each protrusion optionally including one or more electrodes disposed along its length. The one or more protrusions may extend “out of plane” from/relative to a planar region of the neural device 100 (e.g., the thinned silicon wafer), and the one or more protrusions can be moved through (e.g., punched through) the dry sheet of biocompatible material. In this way, the sheet of biocompatible material may coat or surround the planar region of the neural device 100 (e.g., when the dry sheet of biocompatible material is hydrated). In some embodiments, the one or more protrusions including one or more electrodes may be configured to be disposed through a surface of the brain (e.g., to target neurons at different depths under the surface).

In some embodiments, the thinned substrate 140 may be thinned such that the neural device 100 is flexible and can form to a surface of a brain. In some embodiments, the neural device 100 (e.g., the thinned substrate 140 and any encapsulation layers 150) may have a total thickness in a range of about 2 micrometers (μm) to about 1 mm, inclusive of all ranges and subranges therebetween. In some embodiments, the neural device 100 may have a total thickness in a range of about 4 μm to about 12 μm, inclusive of all ranges and subranges therebetween. In some embodiments, the neural device 100 may have a total thickness of about 150 μm. In some embodiments, a thickness of a device layer (e.g., the electrode(s) 110 and the encapsulation layers or a subset thereof) may be in a range of about 10 nanometers (nm) to about 50 μm, inclusive of all ranges and subranges therebetween. In some embodiments, a thickness of a device layer (e.g., the electrode(s) 110 and the encapsulation layers or a subset thereof) may be in a range of about 600 nanometers (nm) to about 50 μm, inclusive of all ranges and subranges therebetween. In some embodiments, a thickness of a device layer (e.g., the electrode(s) 110 and the encapsulation layers or a subset thereof) may be in a range of about 600 nanometers (nm) to about 2 μm, inclusive of all ranges and subranges therebetween. In some embodiments, the device layer may have a thickness in a range of about 660 nm to about 1.05 μm, inclusive of all ranges and subranges therebetween. In some embodiments, a thickness of the thinned substrate 140 may be in a range of about 1 μm to about 100 μm, inclusive of all ranges and subranges therebetween, or of about 1 μm to about 40 μm, inclusive of all ranges and subranges therebetween. In some embodiments, the neural device 100 may have a Young's Modulus in a range of 9 to 200 GPa.

In some embodiments, the neural implant may be coupled to an additional electrode array structure to increase a number of electrode contacts. For example, the neural implant may be coupled to additional surface electrodes, one or more depth electrodes, a depth electrode array, etc. In some embodiments, the additional electrodes may be coupled to and extend from the neural device 100. For example, one or more surface electrodes may extend from the neural device 100 substantially in plane with the neural device 100. In some embodiments, the additional electrodes may extend non-parallel (e.g., substantially perpendicular or at an angle from) a surface of the neural device 100 configured to contact the brain. For example, one or more depth electrodes may be configured to extend from the surface of the neural device 100 and under a surface of the brain. In some embodiments, one or more neural devices 100 may be coupled to one another or configured to communicate with one another. For example, one or more neural devices 100 may be disposed on different regions of the brain and configured to record from and/or stimulate the different regions of the brain (e.g., in coordination, in parallel, etc.).

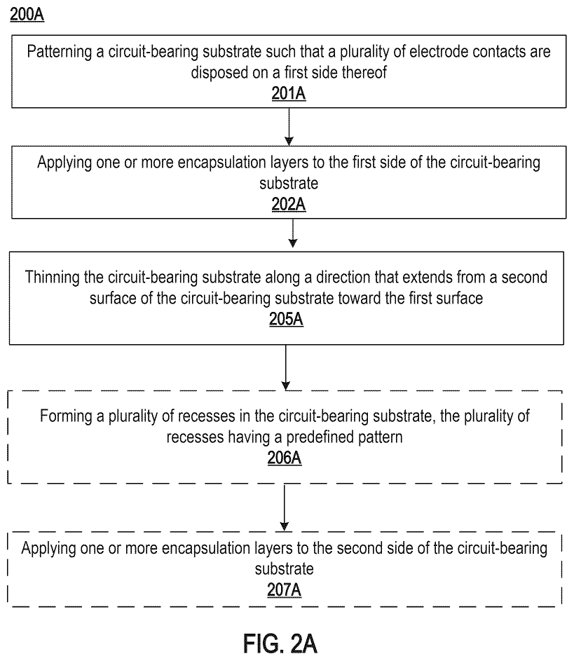

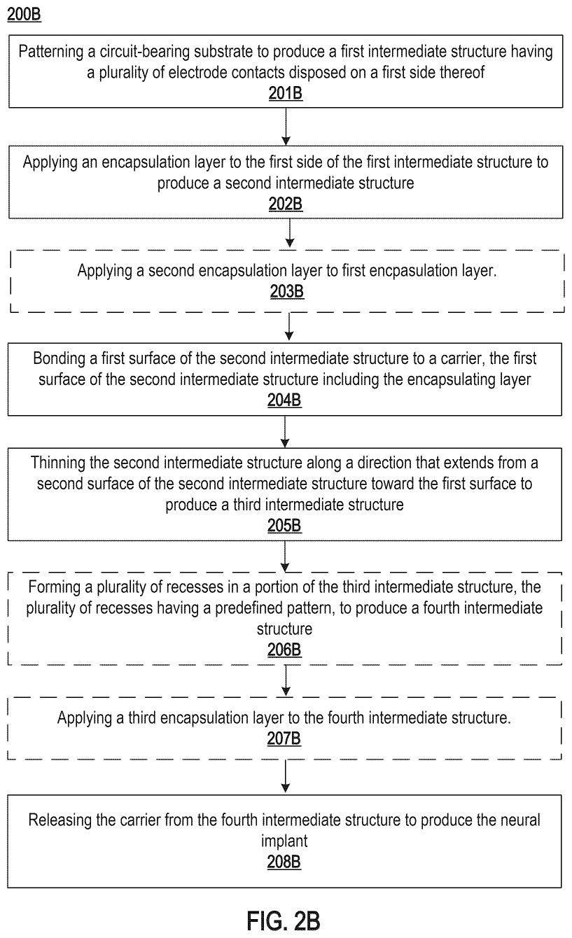

FIG. 2A is a flow chart diagram of a first example method 200A for manufacturing an implantable neural device, according to embodiments. The method 200A may include depositing a conductive material(s) on one or more sides of a substrate. The method 200A may include patterning a circuit-bearing substrate (e.g., a silicon-based wafer) such that a plurality of electrode contacts is disposed on a first side thereof, at 201A. In some embodiments, patterning can include, for example, sputtering, electron beam (“e-beam”) evaporation, electroplating, electroless plating, lithography, photolithography, plasma etching, wet etching, reactive-ion etching, one or more lift-off processes, or a suitable combination thereof, as described in further detail in FIGS. 2B-4H. In some embodiments, the electrode contacts may be patterned on the substrate such that the electrode contacts are disposed in a predetermined pattern or arrangement. In some embodiments, the electrode contacts may be arranged in a uniform arrangement (e.g., a grid). In some embodiments, the electrode contacts may be arranged in a non-uniform or irregular arrangement.

At 202A, the method 200A may include applying one or more encapsulation layers to the first side of the circuit-bearing substrate. In some embodiments, applying the encapsulation layer may include depositing an encapsulant followed by patterning the deposited encapsulant. In some embodiments, the encapsulation layer may be deposited using any suitable method such as physical vapor deposition (CVD), chemical vapor deposition (CVD), atomic layer deposition, pulsed laser deposition, sputtering, or any suitable combination thereof. In some embodiments, a first encapsulation layer may be deposited on the first side of the circuit-bearing substate. In some embodiments, a second encapsulation layer may be disposed on the first encapsulation layer. In some embodiments, a plurality of encapsulation layers may be disposed on the first side of the circuit-bearing substrate. In some embodiments, the encapsulation layers may include the same material. In some embodiments, the encapsulation layers may include different materials. In some embodiments, any or all of the encapsulation layers may include a material such as nitrides, carbides (e.g., silicon carbide), polymers (e.g., parylene-C, polyimide), oxides, (e.g., silicon dioxide) or a suitable combination thereof. In some embodiments, the encapsulation layer closest to the circuit-bearing substrate may include silicon carbide or parylene-C. In some embodiments, the encapsulation layer contacting the circuit-bearing substrate may include silicon carbide or parylene-C. In some embodiments, encapsulation layers not exposed to the external environment may include silicon carbide or parylene-C. In some embodiments, the encapsulation layer furthest from the circuit-bearing substrate (e.g., the layer configured to be exposed to the external environment) may include silicon dioxide.

At 205A, the method 200A may include thinning the circuit-bearing substrate along a direction that extends from a second surface of the circuit-bearing substrate toward the first surface. In some embodiments, thinning may be performed in a manner that produces a substantially planar surface, such that that the thinned surface is free of irregularities. In some embodiments, thinning the circuit-bearing substrate may include at least one of mechanically or chemically thinning the second intermediate structure. In some embodiments, thinning may include chemical mechanical planarization (CMP), deep reactive-ion etching (DRIE), mechanical grinding, wet etching, dry chemical etching (DCE), laser planarization, plasma polishing, or any suitable combination thereof. In some embodiments, the thinning may include bonding (e.g., transfer bonding) the first side of the circuit-bearing substrate (e.g., including the electrode contacts) to a carrier. In some embodiments, the carrier may include a coating. For example, the carrier may include a coating including silicon carbide. In some embodiments, the first side of the circuit-bearing substrate may be bonded to the coating of the carrier using anodic bonding. In some embodiments, the coating of the carrier (e.g., SiC) may be deposited on the first side of the circuit-bearing substrate via chemical vapor deposition (CVD). In some embodiments, thinning the circuit-bearing substrate may be performed until a predefined thickness of the second intermediate structure is reached. For example, mechanical or chemical thinning may be performed for a predetermined amount of time until the predefined thickness is reached. In some embodiments, an etch stop layer may be used such that the predefined thickness is achieved.

The method 200A may optionally include forming a plurality of recesses in the circuit-bearing substrate, the plurality of recesses having a predefined pattern, at 206A. In some embodiments, the predefined pattern may correspond to a number and/or arrangement of electrode contacts on the neural device. In some embodiments, the predefined pattern may be selected based on and/or configured to impart a desired flexibility to the substrate. In some embodiments, the predefined pattern may be a uniform pattern (e.g., grid-like, lattice, etc.). In some embodiments, the predefined pattern may be non-uniform or irregular. For example, a portion of the substrate may include more recesses (e.g., an outer edge/perimeter portion may include more recesses relative to a central portion or a central portion may include more recesses relative to an outer edge/perimeter portion). In some embodiments, the recesses may extend from a first side of the substrate through to the second side of the substrate to form an opening at both ends. In some embodiments, the recesses may not extend all the way through from the first side to the second side of the substrate such that the substrate includes indentations or cavities. The method 200A may optionally include applying one or more encapsulation layers to the second side of the circuit-bearing substrate, at 207A. In some embodiments, one encapsulation layer may be applied to the first side of the circuit-bearing substrate and/or to the second side of the circuit-bearing substrate. In some embodiments, a plurality of encapsulation layers may be applied to the first side of the circuit-bearing substrate and/or second side of the circuit-bearing substrate. In some embodiments, the first side of the circuit-bearing substrate and the second side of the circuit-bearing substrate may include an equal number of encapsulation layers and/or encapsulation material with equal thickness. In some embodiments, the first side of the circuit-bearing substrate and the second side of the circuit-bearing substrate may have a different number of encapsulation layers and/or encapsulation material with different thickness. In some embodiments, the one or more encapsulation layers on the first side of the circuit-bearing substrate and the second side of the circuit-bearing substrate may be the same. In some embodiments, the one or more encapsulation layers on the first side of the circuit-bearing substrate and the second side of the circuit-bearing substrate may be different.

FIG. 2B is a flow chart diagram of a second example method 200B for manufacturing an implantable neural device, according to embodiments. The method 200B may include patterning a circuit-bearing substrate (e.g., a silicon-based wafer) to produce a first intermediate structure having a plurality of electrode contacts disposed on a first side thereof, at 201B. In some embodiments, patterning can include performing metallization (e.g., sputtering, e-beam evaporation, electroplating, electroless plating) of the circuit-bearing substrate and/or lithography and/or a lift-off process. In some embodiments, patterning can include, for example, sputtering, e-beam evaporation, electroplating, electroless plating, lithography, photolithography, plasma etching, wet etching, reactive-ion etching, lift-off processes, or a suitable combination thereof. In some embodiments, the electrode contacts may include materials such as platinum, iridium, ruthenium oxide, and/or iridium oxide, or any suitable material such as those described in FIG. 1. At 202B, the method may include applying an encapsulation layer to the first side of the first intermediate structure to produce a second intermediate structure. In some embodiments, applying the encapsulation layer may include depositing an encapsulant followed by patterning the deposited encapsulant (e.g., to form openings in the encapsulant). In some embodiments, the encapsulation layer may be deposited using any suitable method such as physical vapor deposition (CVD), chemical vapor deposition (CVD), atomic layer deposition, pulsed laser deposition, sputtering, or any suitable combination thereof. In some embodiments, the encapsulation layer may be deposited using CVD. In some embodiments, the patterning the deposited encapsulant may include using plasma etching. In some embodiments, patterning the deposited encapsulant may ensure the encapsulation layer does not cover the electrode contacts. In some embodiments, the encapsulation layer may include a first material including at least one of silicon carbide or parylene-C. In some embodiments, the silicon carbide may include amorphous silicon carbide. In some embodiments, the silicon carbide may include polycrystalline silicon carbide.

In some embodiments, the encapsulation layer may be a first encapsulation layer, and the method 200B may optionally include applying a second encapsulation layer to the first side of the second intermediate structure on the first encapsulation layer, at 203B. In some embodiments, the second encapsulation layer may include a second material different from the first material. In some embodiments, the second material may include an oxide. In some embodiments, the second material may include silicon dioxide. In some embodiments, the second encapsulation layer may be patterned (e.g., using plasma etching). In some embodiments, the second encapsulation layer may be patterned such that at least a portion of the electrode contacts are not covered or encapsulated. In some embodiments, the second encapsulation material may be applied by transfer bonding using a silicon on insulator (SOI) wafer. In some embodiments, both the first encapsulation layer and the second encapsulation layer may be patterned simultaneously. In some embodiments, a thickness of the first encapsulation layer may be in a range of about 0.5 μm to about 10 μm. In some embodiments, a thickness of the second encapsulation layer may be in a range of about 0.5 μm to 10 about μm. In some embodiments, the first encapsulation layer may have a first thickness and the second encapsulation layer may have a second thickness less than the first thickness.

In some embodiments, the method 200B may optionally include transfer bonding the second intermediate structure to a carrier such that backside thinning of the circuit-bearing substrate can be performed. For example, the method 200B may include bonding a first surface of the second intermediate structure including the electrodes and the encapsulating layer (e.g., the first encapsulating layer and/or the second encapsulating layer) to a carrier, at 204B. In some embodiments, the carrier includes at least one of quartz, glass, metal, silicon, and/or ceramic. In some embodiments, the carrier may include a coating. For example, the carrier may include a coating including silicon carbide. In some embodiments, the first side of the circuit-bearing substrate may be bonded to the coating of the carrier using anodic bonding. In some embodiments, the coating of the carrier (e.g., SiC) may be deposited on the first side of the circuit-bearing substrate via chemical vapor deposition (CVD). At 205B, the method 200B includes thinning the second intermediate structure along a direction that extends from a second surface of the second intermediate structure toward the first surface to produce a third intermediate structure (e.g., the thinned substrate 140). The second surface of the second intermediate structure may be opposite the first surface. In other words, thinning may be performed on a surface of the substrate opposite a surface including the electrodes and first and/or second encapsulation layer(s). In some embodiments, thinning the second intermediate structure may include at least one of mechanically or chemically thinning the second intermediate structure. In some embodiments, thinning may include chemical mechanical planarization (CMP), deep reactive-ion etching (DRIE), mechanical grinding, wet etching, dry chemical etching (DCE), laser planarization, plasma polishing, or any suitable combination thereof. In some embodiments, CMP and/or DRIE can be performed to remove silicon from the second side of the second intermediate structure can be performed to remove silicon from the second side of the second intermediate structure. In some embodiments, thinning the second intermediate structure may be performed until a predefined thickness of the second intermediate structure is reached. For example, mechanical or chemical thinning may be performed for a predetermined amount of time until the predefined thickness is reached. In some embodiments, an etch stop layer may be used such that the predefined thickness is achieved.

In some embodiments, the method 200B may optionally include forming a plurality of recesses having a predefined pattern in a portion of the third intermediate structure to produce a fourth intermediate structure, at 206B. Alternatively or additionally to applying the second encapsulation layer to the first encapsulation layer, the method 200B may optionally include applying a third encapsulation layer to the fourth intermediate structure (e.g., on the second side opposite the carrier) prior to releasing the carrier from the fourth intermediate structure, at 207B. Therefore, the neural device may include encapsulation material on both the first side and the second side thereof. In some embodiments, the encapsulation material may coat all sides of the recesses. In some embodiments, the third encapsulation layer may be deposited similarly to the first and/or second encapsulation layers. In some embodiments, applying the third encapsulation layer to the fourth intermediate structure may include patterning (e.g., via plasma etching) the third encapsulation layer. The third encapsulation layer may have a third thickness. In some embodiments the third thickness may be the same as the first thickness. In some embodiments the third thickness of the third encapsulation layer may be in a range of 100 nm to 10 μm. In some embodiments, the third encapsulation layer may include the first material (e.g., parylene-C, silicon carbide, alumina, or a combination thereof). At 208B, the method 200B includes releasing the carrier from the fourth intermediate structure to produce the neural implant.

Method 200B may be similar to method 200A (e.g., may include one or more aspects thereof), and as such, certain details of the method may not be described herein with respect to FIG. 2B. It should be appreciated that the methods 200A and 200B may be performed in any suitable order. For example, application of all of the encapsulation layers may occur after thinning of the substrate. In another example, the substrate may be thinned prior to disposing the electrode contacts on a surface thereof. In another example, the recesses may be formed in the substrate prior to disposing the electrode contacts on a surface thereof. Any and all variations of methods 200A and 200B should be considered within the scope of this application.

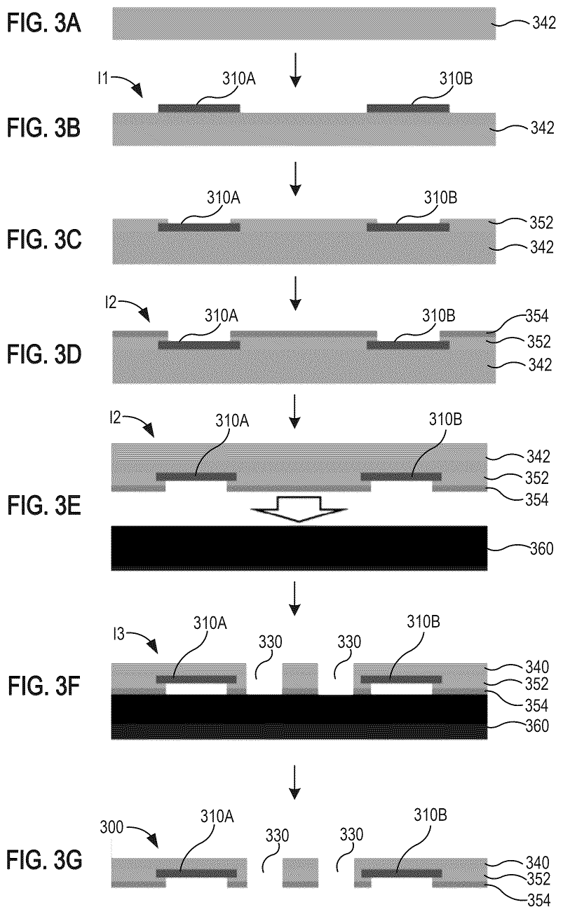

FIGS. 3A-3G show a method of manufacturing an implantable neural device 300, according to embodiments. FIG. 3A shows a silicon (e.g., a complementary metal-oxide-semiconductor (CMOS)) wafer 342 including microelectronics integrated therein. The silicon wafer may be received from a foundry including the microelectronics. The method may include patterning and/or metallization of the silicon wafer 342 (e.g., sputtering and/or lift-off processes) to form a first intermediate structure I1 including at least a pair of electrodes 310A, 310B on a first side thereof, as shown in FIG. 3B. The electrodes 310A, 310B may include platinum, iridium, and/or iridium oxide. As shown in FIG. 3C, a first encapsulation layer 352 may be applied to the first side of the first intermediate structure I1. In some embodiments, the first encapsulation layer 352 may be deposited using CVD and patterned using plasma etching. In some embodiments, at least a portion of the electrodes 310A, 310B are not covered. In some embodiments, the first encapsulation layer 352 may include parylene-C, silicon carbide, and/or a combination thereof. In some embodiments, a second encapsulation layer 354 may be disposed on the first encapsulation layer 352 to form the second intermediate structure I2, as shown in FIG. 3D. The second encapsulation layer 354 may include silicon dioxide. In some embodiments, the second encapsulation layer 354 may be transfer bonded from a SOI wafer and patterned using plasma etching. In some embodiments, at least a portion of the electrodes 310A, 310B are not covered. As shown in FIG. 3E, a first surface (e.g., the surface including the electrodes and the encapsulation layers 352, 354) of the second intermediate structure I2 may be bonded to a carrier 360 (e.g., a carrier including quartz, glass, various metals, silicon, ceramic, etc.). A second side (e.g., not including the electrodes 310A, 310B and encapsulation layers 352, 354) of the second intermediate structure I2 may be thinned to create a thinned substrate 340. Thinning may occur in a direction from the second side toward the first side of the second intermediate structure I2. As shown in FIG. 3F, a plurality of recesses 330 may be formed in the second intermediate structure I2 to form a third intermediate structure I3. In some embodiments, the recesses 330 may be formed by etching the third intermediate structure I3 from the second side toward the first side. In some embodiments, the recesses 330 may extend through at least a portion of the substrate. The recesses 330 may be formed between adjacent electrodes 310A, 310B. As shown, the third intermediate structure I3 includes 2 recesses 330 between the electrodes 310A, 310B. In some embodiments, the recesses 330 may be arranged such that each recess does not align or overlap with an electrode contact 310A, 310B. As shown in FIG. 3G, the third intermediate structure I3 can be released from the carrier 360 to create the neural device 300. The method shown in FIGS. 3A-3G may be substantially similar to the method 200, and therefore, certain details are not described with respect to FIGS. 3A-3G.

FIGS. 4A-3H show a method of manufacturing an implantable neural device 400, according to embodiments. FIG. 4A shows a silicon (e.g., a complementary metal-oxide-semiconductor (CMOS)) wafer 442 including microelectronics integrated therein. The method includes patterning and/or metallization of the silicon wafer 442 (e.g., sputtering with etch-back and/or lift-off processes) to form a first intermediate structure I1 including at least a pair of electrodes 410A, 410B on a first side thereof, as shown in FIG. 4B. The electrodes 410A, 410B may include or be similar to electrodes 110 and 310A, 310B. As shown in FIG. 4C, a first encapsulation layer 452A may be applied to the first side of the first intermediate structure I1. In some embodiments, the first encapsulation layer 452 may be deposited using CVD and patterned using plasma etching. In some embodiments, at least a portion of the electrodes 410A, 410B are not covered. In some embodiments, the first encapsulation layer 352 may include a first material (e.g., parylene-C, silicon carbide, and/or a combination thereof). In some embodiments, a second encapsulation layer 454 (e.g., including silicon dioxide) may be disposed on the first encapsulation layer 452 to form the second intermediate structure I2, as shown in FIG. 4D. In some embodiments, the second encapsulation layer 454 may be transfer bonded from a SOI wafer and patterned using plasma etching. In some embodiments, at least a portion of the electrodes 410A, 410B are not covered. As shown in FIG. 4E, a first surface (e.g., the surface including the electrodes 410A, 410B and the encapsulation layers 452, 454) of the second intermediate structure I2 may be bonded to a carrier 460 (e.g., a carrier including quartz, glass, various metals, silicon, ceramic, etc.). A second side (e.g., not including the electrodes 410A, 410B and encapsulation layers 452, 454) of the second intermediate structure I2 may be thinned to create a thinned substrate 340. Thinning may occur in a direction from the second side toward the first side of the second intermediate structure I2. As shown in FIG. 4F, a plurality of recesses 430 may be formed in the second intermediate structure I2 to form a third intermediate structure I3. The recesses 430 may be formed adjacent to electrodes 410A, 410B. As shown, the third intermediate structure I3 includes 4 recesses 330. A first recess formed on a first side of the electrode 410A, a second recess formed on a second side of the electrode 410A opposite the first side, a third recess formed on a first side of the electrode 410B, and a fourth recess formed on a second side of the electrode 410B opposite the first side.

As shown in FIG. 4G, a third encapsulation layer 452B may be applied to a second side of the third intermediate structure. The third encapsulation layer 452b may include the first material. In some embodiments, the third encapsulation layer 452B may be patterned (e.g., via plasma etching). In some embodiments, at least a portion of the electrodes 410A, 410B is exposed. The third encapsulation layer 452B may coat the recesses 430 such that no portion of the thinned substrate 440 is exposed to an outside environment (e.g., a biological environment inside a patient), or such that at least a portion of the thinned substrate 440 is not exposed to the outside environment. As shown in FIG. 4H, the fourth intermediate structure 14 can be released from the carrier 460 to form the neural device 400. The method shown in FIGS. 4A-4H may be substantially similar to the method 200 and the method shown in FIGS. 3A-3G, and therefore, certain details are not described with respect to FIGS. 3A-3G.

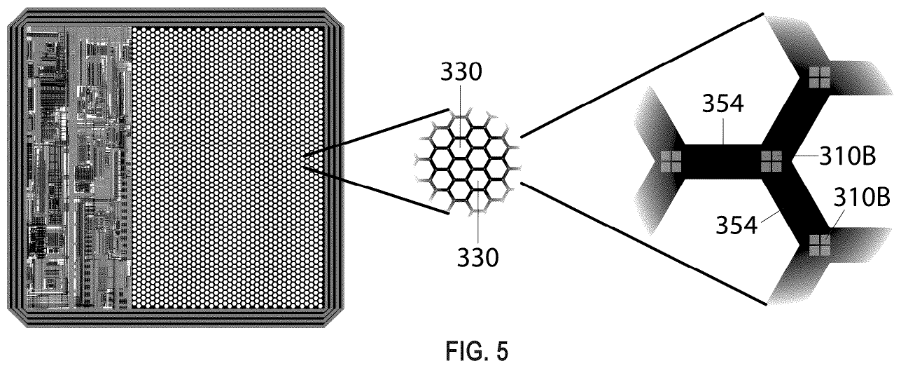

FIG. 5 shows an example implantable neural device manufactured using the method of FIGS. 3A-3G. As shown in FIG. 5, the implantable neural device (depicted on the left side of FIG. 5) includes a plurality of recesses 330 (e.g., similar to the recesses 330 of FIGS. 3A-3G or to the recesses 430 of FIGS. 4A-4H), an encapsulation layer 354 (e.g., similar to the encapsulation layer 354 of FIGS. 3A-3G or to the encapsulation layer 454 of FIGS. 4A-4H), and electrodes 310B (e.g., similar to the electrodes 310B of FIGS. 3A-3G or to the electrodes 410B of FIGS. 4A-4H) which optionally are not covered by, or a subset of which are not covered by, the encapsulation layer 454. Although the electrodes 310B are shown in FIG. 5 as including square arrays of four electrodes each, in other implementations, a different number of electrodes may be included in each cluster/grouping (e.g., two, three, five, six, etc.), a single electrode may be positioned at each vertex/location (or a subset thereof), or any combination of the foregoing may be used (e.g., some vertices/locations may have a single electrode or a first quantity of arrayed or clustered electrodes, while other vertices/locations may have a second quantity of arrayed or clustered electrodes different from the first quantity of arrayed or clustered electrodes). Different electrode shapes (e.g., circular, elliptical, rectangular, triangular, polygonal, etc.) are also contemplated and can be included in a variety of ways, depending on the implementation. For example, some vertices/locations may have square electrodes, while other vertices/locations may have round electrodes.

Various concepts may be embodied as one or more methods, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments. Put differently, it is to be understood that such features may not necessarily be limited to a particular order of execution, but rather, any number of threads, processes, services, servers, and/or the like that may execute serially, asynchronously, concurrently, in parallel, simultaneously, synchronously, and/or the like in a manner consistent with the disclosure. As such, some of these features may be mutually contradictory, in that they cannot be simultaneously present in a single embodiment. Similarly, some features are applicable to one aspect of the innovations, and inapplicable to others.

In addition, the disclosure may include other innovations not presently described. Applicant reserves all rights in such innovations, including the right to embodiment such innovations, file additional applications, continuations, continuations-in-part, divisionals, and/or the like thereof. As such, it should be understood that advantages, embodiments, examples, functional, features, logical, operational, organizational, structural, topological, and/or other aspects of the disclosure are not to be considered limitations on the disclosure as defined by the embodiments or limitations on equivalents to the embodiments. Depending on the particular desires and/or characteristics of an individual and/or enterprise user, database configuration and/or relational model, data type, data transmission and/or network framework, syntax structure, and/or the like, various embodiments of the technology disclosed herein may be implemented in a manner that enables a great deal of flexibility and customization as described herein.

All definitions, as defined and used herein, should be understood to control over dictionary definitions, definitions in documents incorporated by reference, and/or ordinary meanings of the defined terms.

As used herein, in particular embodiments, the terms “about” or “approximately” when preceding a numerical value indicates the value plus or minus a range of 10%. Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While specific embodiments of the present disclosure have been outlined above, many alternatives, modifications, and variations will be apparent to those skilled in the art. Accordingly, the embodiments set forth herein are intended to be illustrative, not limiting. Various changes may be made without departing from the spirit and scope of the disclosure. Where methods and steps described above indicate certain events occurring in a certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

1. A method for manufacturing a neural implant, the method comprising:
patterning a circuit-bearing substrate to produce a first intermediate structure having a plurality of electrode contacts disposed on a first side thereof;
applying an encapsulation layer to the first side of the first intermediate structure to produce a second intermediate structure;
coupling a first surface of the second intermediate structure to a carrier, the first surface of the second intermediate structure including the encapsulating layer;
thinning the second intermediate structure along a direction that extends from a second surface of the second intermediate structure toward the first surface of the second intermediate structure, the second surface opposite the first surface, to produce a third intermediate structure;
forming a plurality of recesses in a portion of the third intermediate structure, the plurality of recesses having a predefined pattern, to produce a fourth intermediate structure; and
releasing the carrier from the fourth intermediate structure to produce the neural implant,
the neural implant not including a hermetic seal.

2. The method of claim 1, wherein applying the encapsulation layer includes depositing an encapsulant followed by patterning the deposited encapsulant.

3. The method of claim 2, wherein patterning the deposited encapsulant includes plasma etching.

4. The method of claim 1, wherein the plurality of electrode contacts includes at least one of tantalum oxide, titanium nitride, PEDOT, carbon nanotubes, gold, platinum, iridium, iridium oxide, ruthenium oxide, or an alloy.

5. The method of claim 1, wherein the encapsulation layer includes at least one of silicon carbide (SiC), doped SiC, polyimide, parylene-C, liquid crystal polymer (LCP), or alumina.

6. The method of claim 5, wherein the SiC includes at least one of amorphous SiC or polycrystalline SiC.

7. The method of claim 1, wherein the encapsulation layer is a first encapsulation layer including a first material, the method further including:
applying a second encapsulation layer over the first encapsulation layer, the second encapsulation layer including a second material different from the first material.

8. The method of claim 7, wherein the second material includes an oxide.

9. The method of claim 7, wherein the second material includes silicon dioxide (SiO₂).

10. The method of claim 1, wherein the carrier comprises at least one of quartz, glass, metal, silicon, or ceramic.

11. The method of claim 1, wherein thinning the second intermediate structure includes at least one of mechanically or chemically thinning the second intermediate structure.

12. The method of claim 1, wherein thinning the second intermediate structure includes:
performing at least one of chemical mechanical planarization (CMP) or reactive-ion etching (RIE) to remove silicon from the second surface of the second intermediate structure.

13. The method of claim 1, wherein thinning the second intermediate structure is performed until a predefined thickness is reached, by one of (1) performing at least one of mechanically or chemically thinning for a predetermined amount of time, or (2) using an etch stop layer.

14. The method of claim 13, wherein the predefined thickness is achieved by at least one of: (1) performing the at least one of mechanically or chemically thinning the second intermediate structure for a predetermined amount of time, or (2) using an etch stop layer.

15. The method of claim 1, wherein patterning the circuit-bearing structure includes:
performing metallization of the circuit-bearing substrate.

16. The method of claim 15, wherein the metallization includes sputtering, and the patterning includes at least one of lithography or a lift-off process.

17. The method of claim 1, wherein the neural implant is configured to wirelessly receive at least one of power or data.

18. The method of claim 1, wherein the encapsulation layer is a first encapsulation layer, the method further comprising applying a second encapsulation layer to the fourth intermediate structure prior to releasing the carrier from the fourth intermediate structure.

19. The method of claim 18, wherein the second encapsulation layer coats the fourth intermediate structure such that at least a portion of the fourth intermediate structure is not exposed to an outside environment.

20. The method of claim 18, wherein the second encapsulation layer includes at least one of silicon carbide (SiC), polyimide, alumina, or parylene-C.

21. The method of claim 1, wherein the encapsulation layer is a first encapsulation layer, the method further comprising, prior to releasing the carrier from the fourth intermediate structure:
applying a second encapsulation layer to the fourth intermediate structure; and
patterning the second encapsulation layer.

22. The method of claim 10, wherein the carrier includes a silicon carbide coating, the coupling the first surface of the second intermediate structure to the carrier further including at least one of:
bonding the first surface of the second intermediate structure to the silicon carbide coating using anodic bonding or depositing the silicon carbide on the first surface of the second intermediate structure using chemical vapor deposition (CVD).

23. The method of claim 1, wherein the forming the plurality of recesses in the portion of the third intermediate structure includes etching the plurality of recesses from the second surface of the third intermediate structure toward the first surface.

24. The method of claim 23, wherein each recess from the plurality of recesses extend through at least a portion of the circuit-bearing substrate.

25. The method of claim 1, wherein the plurality of recesses are arranged in a space between neighboring electrode contacts from the plurality of electrode contacts.
